(19) United States
(12) Patent Application Publication
(10) Pub. No.: US 2026/0174727 A1
(43) Pub. Date: Jun. 25, 2026

(54) APPLICATION OF DI-MYRICETIN DISELENIDE IN DRUG THERAPY FOR TYPE II DIABETES

(71) Applicant: Shanghai Afute Food Technology Co., Ltd., Shanghai (CN)

(72) Inventors: Kunyuan SONG, Shanghai (CN); Weiwei CHEN, Shanghai (CN)

(21) Appl. No.: 19/026,707

(22) Filed: Jan. 17, 2025

(30) Foreign Application Priority Data
Dec. 20, 2024 (CN) 202411896218.X

(51) Int. Cl. A61K 31/353; A61P 3/10

(52) U.S. Cl. CPC A61K 31/353; A61P 3/10

(57) ABSTRACT

The present invention pertains to the field of medical technology, specifically disclosing the pharmaceutical application of dimyricetin-based diselenide in type II diabetes. After four weeks of continuous administration, compared with the model control group, the test substances DMS-ASD and AQ-C-a demonstrated effects that were basically equivalent to the positive control drug, semaglutide injection, in reducing fasting blood glucose levels after 16 hours of fasting in db/db mice, reducing random blood glucose levels in db/db mice, inhibiting postprandial blood glucose elevations in db/db mice, decreasing the HbA1c content of glycated hemoglobin in db/db mice, and influencing body weight and food intake in db/db mice. The pharmaceutical application of dimyricetin-based diselenide in type II diabetes holds great promise.

### TECHNICAL FIELD

This invention pertains to the field of pharmaceutical technology, specifically disclosing the pharmaceutical application of dimyricetin diselenide in the treatment of Type II diabetes.

### BACKGROUND OF RELATED ARTS

Selenium is an essential trace element for the human body, and the Chinese Nutrition Society has listed selenium as one of the 15 essential nutrients for humans. Numerous clinical trials conducted domestically and internationally have demonstrated that selenium deficiency in the human body can cause dysfunction in certain vital organs and even lead to many serious diseases. Appropriate selenium supplementation for individuals with low or deficient selenium levels can not only prevent the occurrence of tumors and liver diseases but also enhance the body's immune capacity, maintain the normal functions of vital organs such as the heart, liver, lungs, and stomach, and prevent the occurrence of geriatric cardiovascular and cerebrovascular diseases. Myricetin is a flavonoid compound widely found in the bark and leaves of waxberry trees. It possesses various pharmacological activities, including anti-inflammatory, anti-tumor, anti-mutagenic, anti-caries, antioxidant properties, and the ability to eliminate free radicals in the body.

Diselenides possess significant antioxidant properties and can mimic the activity of glutathione peroxidase (GSH-PX). Some diselenides also exhibit antitumor, antibacterial, bactericidal, and disinfectant activities. Dimyricetin-yl-diselenide (DMS), with the molecular formula C30H18O16Se2 and a molecular weight of 792.37, appears as a pale yellow powder. It is a novel compound discovered by our company, and we have been conducting continuous research on its pharmaceutical applications. Parts of the research have been patented, such as Chinese patents CN111450088 A and CN 117159532 A, among others.

Type 2 diabetes mellitus (T2DM) is a chronic condition caused by inadequate insulin use or decreased efficiency, primarily manifested as hyperglycemia resulting from insulin resistance and damage to pancreatic beta cells. T2DM affects approximately 437 million people globally and significantly increases the risk of cardiovascular disease, renal disease, liver disease, cancer, and infections. This disease is jointly caused by genetics and environmental factors such as lifestyle, overnutrition, and inadequate physical activity. Often, initial symptoms are mild, and many people may not be diagnosed until complications arise or during routine medical examinations. Accompanying symptoms include fatigue, easy tiredness, susceptibility to colds, mental lethargy, and decreased memory. Currently, the primary treatment for T2DM is pharmacological therapy, supplemented by improvements in lifestyle. However, the commonly used medications at present require long-term administration and have strong dependencies.

### SUMMARY

To address the issues presented in the background technology, this invention discloses the pharmaceutical application of dimyricetin diselenide in the treatment of Type II diabetes. Dimyricetin diselenide demonstrates significant efficacy in treating Type II diabetes and can also address dependency issues.

To achieve the objectives of this invention, the following technical solution is adopted:

The application of dimyricetin diselenide, with the molecular structure shown in formula (1), in the pharmaceutical treatment of Type II diabetes, specifically in the preparation of medications for the treatment of II diabetes. Type

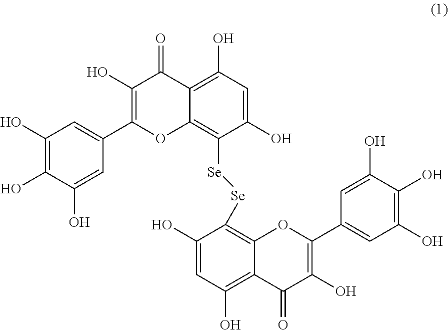 [SMILES: O=c1c(O)c(-c2cc(O)c(O)c(O)c2)oc2c([Se][Se]c3c(O)cc(O)c4c(=O)c(O)c(-c5cc(O)c(O)c(O)c5)oc34)c(O)cc(O)c12]

Furthermore, the application of the dimyricetin diselenide in medicine for type II diabetes is introduced, wherein the dimyricetin diselenide refers to dimyricetin diselenide itself or its solid dispersion formulation.

Furthermore, the application of the dimyricetin diselenide solid dispersion formulation in medicine for type II diabetes is elaborated. This formulation comprises dimyricetin diselenide and a carrier, with the carrier being polyvinyl pyrrolidone K30.

Compared with the existing technology, the beneficial effects of the present invention are:

The present invention discloses the pharmaceutical application of dimyricetin-based diselenide in type II diabetes. After four weeks of continuous administration, compared with the model control group, the test substances DMS-ASD and AQ-C-a demonstrated effects that were basically equivalent to the positive control drug, semaglutide injection, in reducing fasting blood glucose after 16 hours, reducing random blood glucose, inhibiting postprandial blood glucose elevation, decreasing glycosylated hemoglobin HbA1c levels in db/db mice, and in their impact on body weight and food intake in db/db mice. Dimyricetin-based diselenide holds great promise for pharmaceutical application in type II diabetes.

### BRIEF DESCRIPTION OF THE DRAWINGS

FIG. 1 illustrates the effect of the test substance on fasting blood glucose in db/db mice after four consecutive weeks of oral gavage (once a day) in the present invention (Mean±SEM, n=9/10);

FIG. 2-1 depicts the effect of the test substance on random blood glucose in db/db mice after four consecutive weeks of oral gavage (once a day) in the present invention (Mean±SEM, n=9/10);

FIG. 2-2 displays the effect of the test substance on AUC0-24 h of random blood glucose in db/db mice after four consecutive weeks of oral gavage (once a day) in the present invention (Mean±SEM, n=9/10);

FIG. 3-1 shows the effect of the test substance on OGTT in db/db mice after four consecutive weeks of oral gavage (once a day) in the present invention (Mean±SEM, n=9/10);

FIG. 3-2 presents the effect of the test substance on AUC0-180 min of OGTT blood glucose in db/db mice after four consecutive weeks of oral gavage (once a day) in the present invention (Mean±SEM, n=9/10);

FIG. 4 illustrates the effect of the test substance on the level of glycated hemoglobin HbA1c in db/db mice after four consecutive weeks of oral gavage (once a day) in the present invention (Mean±SEM, n=9/10);

FIG. 5 depicts the effect of the test substance on plasma insulin levels in db/db mice after four consecutive weeks of oral gavage (once a day) in the present invention (Mean±SEM, n=9/10);

FIG. 6 displays the effect of the test substance on body weight in db/db mice after four consecutive weeks of oral gavage (once a day) in the present invention (Mean±SEM, n=9/10);

FIG. 7 shows the effect of the test substance on food intake in db/db mice after four consecutive weeks of oral gavage (once a day) in the present invention (Mean±SEM, n=9/10).

### DETAILED DESCRIPTIONS OF EMBODIMENTS

### Case Study 1: In Vivo Pharmacodynamics Experiment

### 1) Experimental Drug

### \textcircled{1} Test Substance

Name of Test Substance 1: DMS-ASD (Polyvinylpyrrolidone K30 Solid Dispersion Formulation of Dimyricetin Diselenide, Preparation Method Refer to CN117379377A)
Source: Shanghai Aiqi Pharmaceutical Technology Co., Ltd.
Appearance: Yellow Powder
Average Molecular Weight: N/A
Specification: N/A
Batch Number: PCS8888-AQ1-DO010
Expiry Date: N/A
Storage Conditions: Sealed and stored at room temperature.
   Name of Test Substance II: AQ-C-a (Dimyricetin Diselenide, Preparation Method Refer to CN111454240B)
   Source: Shanghai Aiqi Pharmaceutical Technology Co., Ltd.
   Appearance: Brown Powder
   Average Molecular Weight: N/A
   Specification: N/A
   Batch Number: 20240315
   Expiration Date: N/A
Storage Conditions: Sealed and stored at room temperature.

### \textcircled{1} Positive Control Drug

Name: Semaglutide Injection (Ozempic®)
Source: Novo Nordisk (China) Pharmaceutical Co., Ltd.
Appearance: Clear and colorless liquid
Average Molecular Weight: 4113.58 Da
Specification: 1.34 mg/mL; 3 mL/vial*1 vial/box
Batch Number: 202206BAG1
Expiry Date: May 2025
Storage Conditions: Store at 2˜8° C., protect from light, and keep tightly closed . . .

### 2) Feeding of Experimental Animals

### \textcircled{1} Animal

Species and strain: db/db mice
Grade: SPF
Gender: Male
Source: Changzhou Cavens Experimental Animal Co., Ltd.
Experimental Animal Quality Certificate No.: 320730240100201187
Experimental Animal Production License No.: SCXK (Jiangsu) 2021-0013
Experimental Animal Use License No.: SYXK (Shanghai) 2020-0038
Number of animals arrived: 45 db/db mice
Age of animals arrived: 8-9 weeks old
Acclimation period: 5-7 days
Animal numbering method: Each cage is equipped with an identity card containing information such as the project number, experimental group, name of the experimenter, animal species, and gender. The mice are marked with lines on their tail roots . . .

### \textcircled{2} Environment

The animal room is maintained at a temperature of 20˜25° C., with a humidity of 40˜70%, and a 12-hour light-dark cycle. Animals are housed in cages with 5 per cage, and the bedding material (corncob bedding, supplied by Suzhou Daichuan Trading Co., Ltd.) is changed three times a week.

### \textcircled{3} Food and Drinking Water

All mice were fed with experimental mouse growth and reproduction feed (sterilized with Co60, purchased from Jiangsu Xie Tong Medicine & Biology Engineering Co., Ltd.), and the water for experimental animals was filtered and sterilized with high pressure.

### \textcircled{4} Animal Selection and Fasting

The animals used in the experiment were maintained in good health. During the experiment, the animals were fasted according to experimental requirements (with water access allowed).

### 3) Main Instruments and Reagents for the Experiment

### \textcircled{1} Main Instruments

| Instrument | Manufacturer | Model |
|---|---|---|
| Pipette | Eppendorf | (10~1000) μL |
| Centrifuge | SIGMA | 3-30KS |
| Microplate reader | MOLECULAR | VersaMax |
| Blood glucose analyzer | DEVICES | StatStrip Xpress |
| Blood glucose meter | Nova Biomedica | Performa |
| Blood glucose test strips | Roche Diabetes Care | ACCU-CHEK Performa- |
| | Roche Diabetes Care | 06454011 |

### \textcircled{2} Main Reagents

| Instrument | Source/Batch Number |
|---|---|
| Rat/Mouse Insulin | Millipore/4149798 |
| Sterile Water for Injection | 24042703A |
| Methylcellulose | F23161471 |
| Phosphate Buffer Saline Pack | XK353083 |
| Anhydrous Glucose | 20230713 |
| K₂EDTA Anticoagulant Tube | 3108859 |

### 4) Experimental Method

### \textcircled{1} Animal Grouping

The mice were purchased and underwent adaptive feeding. After the db/db mice reached the desired blood glucose level (fasting blood glucose ≥16.7 mmol/L after 4 hours of fasting), those with the targeted blood glucose values were selected and randomly stratified into groups based on their blood glucose levels for experimentation. The db/db mice were divided into a total of four groups, with 10 mice in each group. The grouping is as follows:

| | | | | | | Dosage |
|---|---|---|---|---|---|---|
| | | | | Administration | Administration | method, |
| | | Administration | | volume | concentration | frequency, |
| Group | Drug treatment | does | | (mL/kg) | (mg/mL) | and cycle |
| G1: model | solvent | — | | 10 | | PO, QD |
| control group | | | | | | *4 Weeks |
| G2: positive | Semaglutide | 10 nmol/kg | | 5 | 8.23 μg/mL | SC, QD |
| control drug | Injection | (41.14 μg/kg) | | | | *4 Weeks |
| group | (Ozempic ®) | | | | | |
| G3: DMS- | DMS-ASD | 1000 | mg/kg | 10 | 100 | PO, QD |
| ASD group | | | | | | *4 Weeks |
| G4: AQ-C-a | AQ-C-a | 600 | mg/kg | 10 | 60 | PO, QD |

| | |
|---|---|
| group | *4 Weeks |

### \textcircled{2} Drug Formulation

### A. Preparation of Dosage Forms

| | Dose | | Concentration | |
|---|---|---|---|---|
| Medicine | (μg/kg) | | (mg/mL) | Formulate |
| G2: positive | 10 nmol/kg | | 8.23 μg/mL | Pipette 20.0 μL of the positive control |
| control drug | (41.14 μg/kg) | | | drug and add it to 3.236 mL of PBS |
| | | | | solution; |
| G3: DMS-ASD | 1000 | mg/kg | 100 | Weigh an appropriate amount of the test |
| group | | | | substance, add it to a 0.5% MC solution, |
| | | | | mix well, and use it immediately after |
| | | | | preparation; |
| G4: AQ-C-a | 600 | mg/kg | 60 | Weigh an appropriate amount of the test |

| | |
|---|---|
| group | substance, add it to a 0.5% MC solution, |
| | mix well, and use it immediately after |
| | preparation. |

### B. Preparation of 20% Glucose Solution

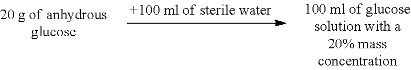

### \textcircled{3} Method

After adaptive feeding, the fasting blood glucose (fasted for 4 hours) of db/db mice was measured weekly until their blood glucose reached the hyperglycemic criterion (fasting blood glucose ≥16.7 mmol/L after a 4-hour fast). Qualified db/db mice were selected for enrollment and stratified randomly into four groups of 10 mice each based on their fasting blood glucose levels: Model Control Group, Positive Control Drug Group, DMS-ASD Group, and AQ-C-a Group.

After grouping, animals in each group were administered with the corresponding drug solution based on their body weights. The route and frequency of drug administration were in accordance with the animal grouping table in \textcircled{1}. This process lasted for approximately 4 consecutive weeks. Animals were fasted for 4 hours before each daily administration and allowed to resume free feeding 1 hour afterward. The first day of drug administration was recorded as D1. During the continuous administration period, fasting blood glucose levels (after 16 hours of fasting) were monitored once a week, while food intake and body weight were monitored twice a week. On D28, random blood glucose levels were measured at 0 h (0-60 minutes before drug administration), 0.5 h, 1 h, 2 h, 4 h, 8 h, 24 h, and 48 h post-administration. Subsequently, after an overnight fast, an OGTT test was conducted. The following day (after another overnight fast), animals were deeply anesthetized with CO2, and blood was collected from the heart for the detection of glycated hemoglobin HbA1c levels (using anticoagulated blood) and plasma insulin levels (using an ELISA kit).

### \textcircled{4} Data Statistics

The experimental data are presented as Mean±SD and analyzed statistically using SPSS 21.0 software. One-way ANOVA is employed for statistical analysis. If the variance is homogeneous, the Tukey test is used for comparison between groups. In cases of heterogeneous variance, the Dunnett's T3 test is applied for comparison between groups. Statistical significance is considered when P<0.05.

### 5) Experimental Result

\textcircled{1} Effect of the Test Substance on the 16-Hour Fasting Blood Glucose Levels in db/db Mice after Continuous Oral Gavage (Once a Day) for Four Weeks

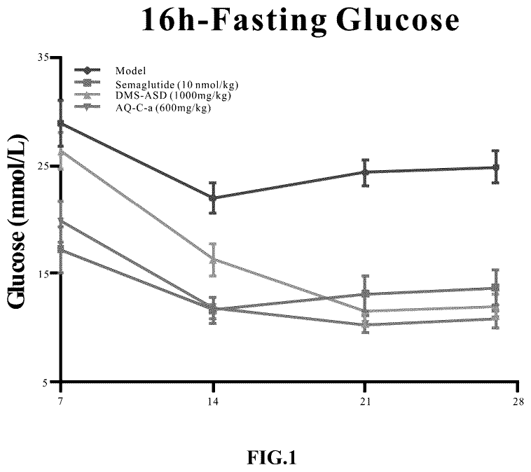

As shown in Table 1 and FIG. 1, during the continuous drug administration period, compared with the model control group, the 16-hour fasting blood glucose levels of db/db mice in the positive control group receiving 10 nmol/kg of semaglutide injection were significantly lower, with statistically significant differences (P<0.01, P<0.001 vs. model control group). In the test group receiving 1000 mg/kg of DMS-ASD, the 16-hour fasting blood glucose levels of db/db mice showed a decreasing trend starting from Day 7 post-administration, which lasted until Day 27 post-administration, and were significantly lower on Day 21 and Day 27 compared with the model control group, with statistically significant differences (P<0.001 vs. model control group). Additionally, in the test group receiving 600 mg/kg of AQ-C-a, the 16-hour fasting blood glucose levels of db/db mice were significantly lower throughout the entire drug administration period compared with the model control group, with statistically significant differences (P<0.05, P<0.001 vs. model control group).

In summary, the test substances DMS-ASD at 1000 mg/kg and AQ-C-a at 600 mg/kg exhibit significant hypoglycemic effects in db/db mice fasted for 16 hours. The pharmacological effect of AQ-C-a at 600 mg/kg is basically equivalent to that of semaglutide injection at 10 nmol/kg. After continuous administration of DMS-ASD at 1000 mg/kg for 21 days, its pharmacological effect approximates that of semaglutide injection.

TABLE 1
The effect of the test substance, administered orally by gavage once a day for four
consecutive weeks, on fasting blood glucose levels in db/db mice (Mean ± SD)

| | | 16 h-Fasting Glucose (mmol/L) | | | |
|---|---|---|---|---|---|
| Group | N | Day 7 | Day 14 | Day 21 | Day 27 |
| Model | 10 | 28.91 ± 6.74 | 22 ± 4.66 | 24.36 ± 3.8 | 24.85 ± 4.7 |
| Semaglutide (10 | 10 | 17.21 ± 6.65** | 11.62 ± 3.98*** | 13.12 ± 5.22*** | 13.72 ± 5.17*** |
| nmol/kg) | | | | | |
| DMS-ASD (1000 | 9 | 26.31 ± 5.33 | 16.31 ± 4.62 | 11.57 ± 4.67*** | 12 ± 3.44*** |
| mg/kg) | | | | | |
| AQ-C-a (600 | 9 | 19.81 ± 6.22* | 11.86 ± 3.12*** | 10.32 ± 2.56*** | 10.8 ± 2.22*** |
| mg/kg) | | | | | |

[symbol]

: P* < 0.05, P** < 0.01, P*** < 0.001 compared with the model control group; Animals in G3-4 and G4-8 died on Day 27 and Day 22, respectively, with renal cysts visible upon dissection.

\textcircled{2} Effect of Four Weeks of Consecutive Daily Oral Gavage Administration of the Test Substance on Random Blood Glucose in db/db Mice

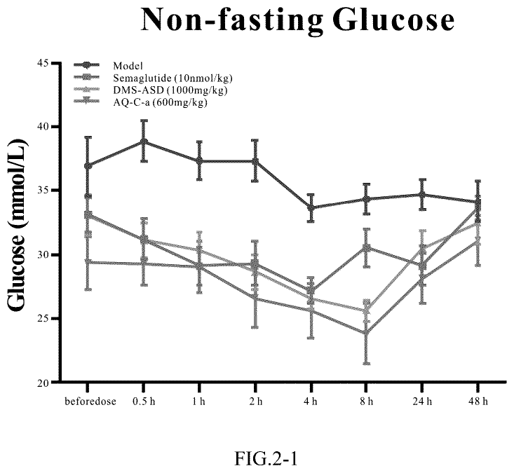

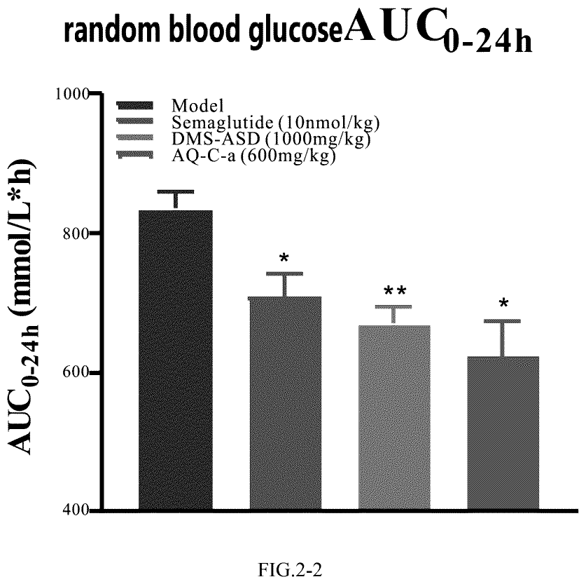

After four weeks of consecutive daily oral gavage administration of the test substance to db/db mice, random blood glucose levels were monitored within 48 hours post-dosing. As shown in Table 2, FIG. 2-1, and FIG. 2-2, compared to the model control group, the DMS-ASD test substance group at 1000 mg/kg exhibited a decreasing trend in random blood glucose levels in db/db mice starting from 0.5 hours post-dosing, which lasted up to 24 hours post-dosing. Statistically significant differences were observed at 0.5, 1, 2, 4, and 8 hours post-dosing (P<0.05, P<0.01, P<0.001 vs. model control group). Similarly, the AQ-C-a test substance group at 600 mg/kg showed a decreasing trend in random blood glucose levels starting from 0.5 hours post-dosing, which persisted up to 48 hours post-dosing, with statistically significant differences at 0.5, 1, 2, 4, and 8 hours post-dosing (P<0.05, P<0.01 vs. model control group). The positive control group receiving semaglutide injection at 10 nmol/kg demonstrated a decreasing trend in random blood glucose levels starting from 0.5 hours post-dosing, lasting up to 24 hours post-dosing, with statistically significant differences at 0.5, 1, 2, and 4 hours post-dosing (P<0.05, P<0.01 vs. model control group). The AUC0-24 h for random blood glucose in the DMS-ASD test substance group at 1000 mg/kg, the AQ-C-a test substance group at 600 mg/kg, and the semaglutide injection positive control group at 10 nmol/kg were significantly lower than that of the model control group, with statistical significance (P<0.05, P<0.01 vs. model control group).

In summary, both the test substances, DMS-ASD at 1000 mg/kg and AQ-C-a at 600 mg/kg, can reduce the random blood glucose levels in db/db mice, with a slightly superior efficacy compared to the positive control drug, semaglutide injection, at 10 nmol/kg. However, there is no statistically significant difference.

TABLE 2
The effect of continuous four-week oral gavage administration (once a day) of
the test substance on random blood glucose levels in db/db mice (Mean ± SD)

| | | | | | | | | | | AUC_{0-24 h} |
|---|---|---|---|---|---|---|---|---|---|---|
| | | Non-Fasting Glucose (Day 28) | | | | | | | | (mmol/ |
| Group | N | 0 h | 0.5 h | 1 h | 2 h | 4 h | 8 h | 24 h | 48 h | L*min) |
| Model | 10 | 36.92 ± | 38.85 ± | 37.33 ± | 37.29 ± | 33.63 ± | 34.35 ± | 34.7 ± | 34.12 ± | 834.58 ± |
| | | 7.31 | 5.05 | 4.61 | 5.04 | 3.28 | 3.6 | 3.73 | 4.98 | 83.02 |
| Semaglutide | 10 | 33.12 ± | 31.2 ± | 29.09 ± | 29.28 ± | 27.17 ± | 30.51 ± | 29.11 ± | 33.67 ± | 709.11 ± |
| (10 | | 4.26 | 5.22* | 4.67** | 5.41* | 3.03** | 4.76 | 4.9 | 3.01 | 97.73* |
| nmol/kg) | | | | | | | | | | |
| DMS- | 9 | 33.07 ± | 31.09 ± | 30.38 ± | 28.64 ± | 26.54 ± | 25.63 ± | 30.46 ± | 32.44 ± | 669.17 ± |
| ASD | | 4.78 | 4.04* | 4.2* | 4.01** | 2.38*** | 2.47*** | 4.28 | 4.9 | 70.8** |
| (1000 | | | | | | | | | | |
| mg/kg) | | | | | | | | | | |
| AQ-C-a | 9 | 29.32 ± | 29.28 ± | 29.01 ± | 26.6 ± | 25.59 ± | 23.79 ± | 28.1 ± | 31.06 ± | 623.08 ± |
| (600 | | 6.37 | 5.12** | 6.07* | 7.07* | 6.51* | 7.05** | 5.86 | 5.93 | 149.18* |
| mg/kg) | | | | | | | | | | |

[symbol]

: P < 0.05, P < 0.01, P < 0.001 compared with the model control group; Animals in G3-4 and G4-8 died on Day 27 and Day 22 respectively, and renal cysts were visible upon dissection.

\textcircled{3} the Effect of the Test Substance, Administered Orally by Gavage Once a Day for Four Consecutive Weeks, on the OGTT in db/db Mice

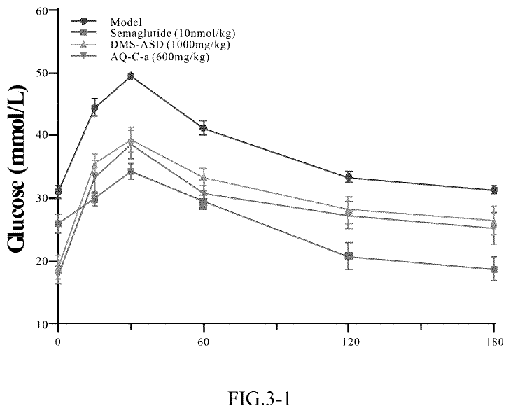

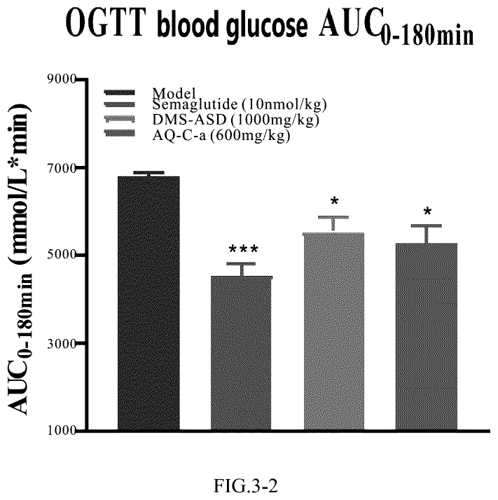

As shown in Table 3, FIGS. 3-1, and 3-2, compared with the model control group, the db/db mice in the 1000 mg/kg DMS-ASD group, the 600 mg/kg AQ-C-a group, and the 10 nmol/kg positive control drug semaglutide injection group exhibited a reduced blood glucose elevation effect after glucose administration.

Based on the blood glucose AUC0-180 min from the OGTT, it is observed that, compared with the model control group, the AUC0-180 min values for the 1000 mg/kg DMS-ASD group, the 600 mg/kg AQ-C-a group, and the 10 nmol/kg positive control group (semaglutide injection) are significantly lower, with statistical differences (P<0.05, P<0.001 vs model control group). The efficacy of 1000 mg/kg DMS-ASD and 600 mg/kg AQ-C-a in inhibiting the post-glucose blood glucose increase in db/db mice is slightly weaker than that of the 10 nmol/kg positive control semaglutide injection, but this difference is not statistically significant.

TABLE 3
The effect of continuous four-week oral gavage administration (once
per day) of the test substance on the OGTT in db/db mice (Mean ± SD)

| | | OGTT | | | | | | |
|---|---|---|---|---|---|---|---|---|
| | | 0 | 15 | 30 | 60 | 120 | 180 | AUC_{0-180 min}(mmol/ |
| Group | N | min | min | min | min | min | min | L*min) |
| Model | 10 | 31.03 ± | 44.41 ± | 49.54 ± | 41.18 ± | 33.4 ± | 31.38 ± | 6812 ± |
| | | 2.93 | 4.19 | 1.05 | 3.39 | 2.31 | 2.03 | 254.27 |
| Semaglutide | 10 | 25.99 ± | 29.87 ± | 34.31 ± | 29.41 ± | 20.76 ± | 18.66 ± | 4543.55 ± |
| (10 | | 4.42 | 3.56*** | 3.48*** | 3.2*** | 6.29** | 5.85*** | 807.92*** |
| nmol/kg) | | | | | | | | |
| DMS- | 9 | 18.99 ± | 35.44 ± | 39.37 ± | 33.38 ± | 28.14 ± | 26.37 ± | 5541.5 ± |
| ASD | | 5.71*** | 4.62** | 6.2*** | 4.39** | 6.48 | 6.76 | 975.88* |
| (1000 | | | | | | | | |
| mg/kg) | | | | | | | | |
| AQ- | 9 | 17.68 ± | 33.39 ± | 38.66 ± | 30.71 ± | 27.32 ± | 25.16 ± | 5279.17 ± |
| C-a | | 4.04*** | 7.77* | 6.75*** | 7.32* | 6.64 | 7.55 | 1204.61* |
| (600 | | | | | | | | |
| mg/kg) | | | | | | | | |

Note:
P < 0.05, P < 0.01, P < 0.001 compared with the model control group; Animals in G3-4 and G4-8 died on Day 27 and Day 22, respectively, with renal cysts visible upon dissection.

\textcircled{4} Effect of the Test Substance on the Glycated Hemoglobin HbA1c Content in db/db Mice after Four Consecutive Weeks of Oral Gavage (Once a Day)

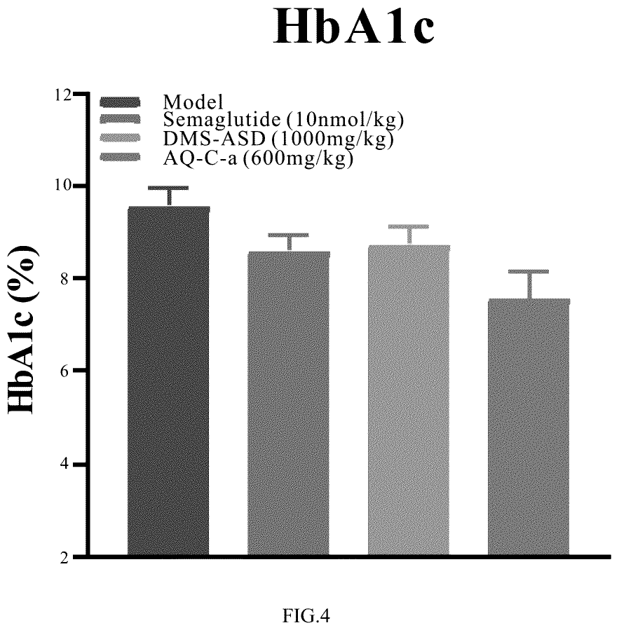

As shown in Table 4 and FIG. 4, the results of glycated hemoglobin HbA1c indicate that the test substances DMS-ASD at 1000 mg/kg, AQ-C-a at 600 mg/kg, and the positive control drug semaglutide injection at 10 nmol/kg all exhibit a trend of reducing the HbA1c content in db/db mice. However, there are no statistically significant differences.

TABLE 4
Effect of Continuous Administration of the Test Substance
by Oral Gavage (Once a Day) for Four Weeks on the Glycated
Hemoglobin (HbA1c) Level in db/db Mice (Mean ± SD)

| Group | N | HbA1c % |
|---|---|---|
| Model | 10 | 9.59 ± 1.19 |
| Semaglutide (10 nmol/kg) | 10 | 8.57 ± 1.03 |
| DMS-ASD (1000 mg/kg) | 9 | 8.72 ± 1.13 |
| AQ-C-a (600 mg/kg) | 9 | 7.54 ± 1.71 |

Note:
Animals in G3-4 and G4-8 died on Day 27 and Day 22 respectively, and renal cysts were observed during dissection.

\textcircled{5} the Effect of Continuous Four-Week Oral Gavage Administration of the Test Substance (Once a Day) on Plasma Insulin Levels in db/db Mice

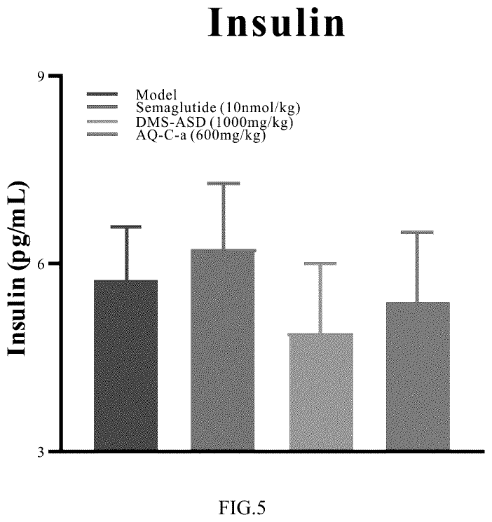

As shown in Table 5 and FIG. 5, after four weeks of continuous oral gavage administration of the test substances (once a day), the results of plasma insulin levels indicated that compared with the model control group, the db/db mice in the 10 nmol/kg positive control group receiving semaglutide injection exhibited a trend of increased plasma insulin levels, although the difference was not statistically significant. However, no increase in plasma insulin levels was observed in the db/db mice from the 1000 mg/kg DMS-ASD group and the 600 mg/kg AQ-C-a group of test substances.

In summary, the positive control drug semaglutide injection at a dose of 10 nmol/kg exhibits a tendency to promote insulin secretion in db/db mice. However, the test substances DMS-ASD at 1000 mg/kg and AQ-C-a at 600 mg/kg do not significantly increase insulin secretion in db/db mice.

TABLE 5
Effect of continuous oral gavage administration of
the test substance (once per day) for four weeks
on plasma insulin levels in db/db mice (Mean ± SD)

| Group | N | Insulin (ng/mL) |
|---|---|---|
| Model | 9 | 5.75 ± 2.52 |
| Semaglutide (10 nmol/kg) | 10 | 6.25 ± 3.34 |
| DMS-ASD (1000 mg/kg) | 9 | 4.9 ± 3.35 |
| AQ-C-a (600 mg/kg) | 9 | 5.42 ± 3.31 |

Note:
Animals in G3-4 and G4-8 died on Day 27 and Day 22 respectively, with renal cysts observed during dissection. Animal in G1-4 died on Day 37, with renal cysts also observed during dissection.

\textcircled{6} Effect of Daily Oral Gavage (Once a Day) of the Test Substance for Four Consecutive Weeks on the Body Weight of db/db Mice

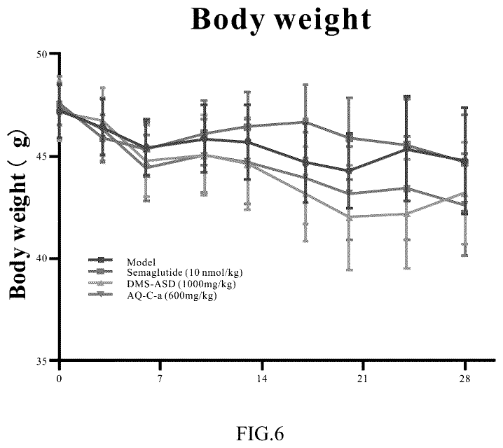

As shown in Table 6 and FIG. 6, after four weeks of continuous administration, compared with the model control group, there was little fluctuation in body weight among db/db mice in the positive control group treated with 10 nmol/kg of Semaglutide Injection. The db/db mice in the test groups of 1000 mg/kg DMS-ASD and 600 mg/kg AQ-C-a exhibited a trend of decreased body weight, but neither difference reached statistical significance.

TABLE 6
Effect of Four Consecutive Weeks of Daily Oral Gavage Administration (1 time/day)
of the Test Substance on the Body Weight of db/db Mice (Mean ± SD)

| | | Body weight (g) | | | | | | | | |
|---|---|---|---|---|---|---|---|---|---|---|
| | | Before | | | | | | | | |
| Group | N | dose | Day 3 | Day 6 | Day 10 | Day 13 | Day 17 | Day 20 | Day 24 | Day 28 |
| Model | 10 | 47.17 ± | 46.39 ± | 45.42 ± | 45.83 ± | 45.69 ± | 44.65 ± | 44.27 ± | 45.34 ± | 44.76 ± |
| | | 4.22 | 4.36 | 4.4 | 5.21 | 5.78 | 6.18 | 5.86 | 8.02 | 8.16 |
| Semaglutide | 10 | 47.53 ± | 45.85 ± | 45.31 ± | 46.11 ± | 46.43 ± | 46.66 ± | 45.86 ± | 45.54 ± | 44.66 ± |
| (10 | | 3.27 | 3.74 | 4.25 | 4.95 | 5.43 | 5.7 | 6.31 | 7.09 | 7.38 |
| nmol/kg) | | | | | | | | | | |
| DMS- | 9 | 47.15 ± | 46.71 ± | 44.74 ± | 45.01 ± | 44.6 ± | 43.14 ± | 41.99 ± | 42.16 ± | 43.19 ± |
| ASD | | 4.55 | 5.23 | 5.58 | 6.22 | 7.09 | 7.41 | 8.19 | 8.46 | 7.52 |
| (1000 | | | | | | | | | | |
| mg/kg) | | | | | | | | | | |
| AQ-C-a | 9 | 47.32 ± | 46.31 ± | 44.39 ± | 45.01 ± | 44.71 ± | 43.93 ± | 43.17 ± | 43.41 ± | 42.61 ± |
| (600 | | 4.99 | 5 | 5.16 | 5.72 | 6.54 | 7.09 | 7.18 | 7.61 | 7.41 |
| mg/kg) | | | | | | | | | | |

Note:
Animals in G3-4 and G4-8 died on Day 27 and Day 22, respectively. Renal cysts were visible upon dissection.

\textcircled{7} Effect of Continuous Four-Week Oral Gavage Administration of the Test Substance (Once a Day) on Food Intake in db/db Mice

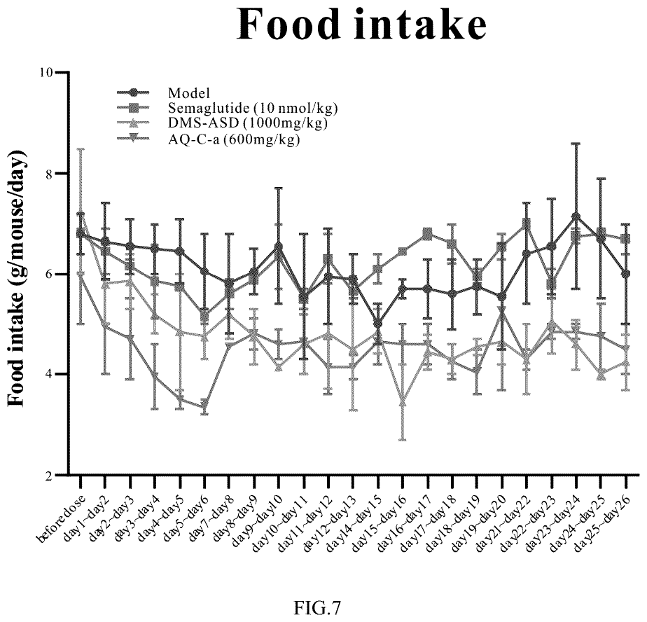

As shown in Table 7, Continued Table 7-1, Continued Table 7-2, and FIG. 7, after four weeks of continuous drug administration, compared with the model control group, there was no significant change in food intake among db/db mice in the positive control group receiving 10 nmol/kg of semaglutide injection. In contrast, the food intake of db/db mice in the test groups receiving 1000 mg/kg of DMS-ASD and 600 mg/kg of AQ-C-a gradually decreased and reached the lowest level on Day 7, thereafter maintaining at a relatively low level. Before Day 7, the db/db mice in the test group receiving 600 mg/kg of AQ-C-a exhibited a greater decrease in food intake compared to those in the test group receiving 1000 mg/kg of DMS-ASD . . .

TABLE 7
Effect of Continuous Four-Week Administration of the Test Substance
by Oral Gavage (Once a Day) on Food Intake in db/db Mice (Mean ± SD)

| | | Food Intake (g/mouse/day) | | | | | | | |
|---|---|---|---|---|---|---|---|---|---|
| | | before | day 1~ | day 2~ | day 3~ | day 4~ | day 5~ | day 7~ | day 8~ |
| Group | N | dose | day 2 | day 3 | day 4 | day 5 | day 6 | day 8 | day 9 |
| Model | 10 | 6.78 ± | 6.68 ± | 6.53 ± | 6.47 ± | 6.45 ± | 6.05 ± | 5.77 ± | 6.05 ± |
| | | 0.54 | 1.04 | 0.76 | 0.74 | 0.92 | 1.12 | 1.4 | 0.6 |
| Semaglutide | 10 | 6.79 ± | 6.49 ± | 6.17 ± | 5.85 ± | 5.76 ± | 5.15 ± | 5.56 ± | 5.92 ± |
| (10 | | 0.61 | 0.64 | 0.23 | 1 | 0.03 | 0.15 | 0.42 | 0.13 |
| nmol/kg) | | | | | | | | | |
| DMS- | 9 | 7.25 ± | 5.8 ± | 5.84 ± | 5.2 ± | 4.85 ± | 4.73 ± | 5.16 ± | 4.74 ± |
| ASD | | 1.8 | 1.17 | 0.76 | 0.5 | 1.57 | 0.61 | 0.71 | 0.79 |
| (1000 | | | | | | | | | |
| mg/kg) | | | | | | | | | |
| AQ-C-a | 9 | 5.93 ± | 4.95 ± | 4.7 ± | 3.94 ± | 3.51 ± | 3.38 ± | 4.57 ± | 4.79 ± |
| (600 | | 1.34 | 1.29 | 1.17 | 0.94 | 0.3 | 0.23 | 0.04 | 0.43 |
| mg/kg) | | | | | | | | | |
| | | Food Intake (g/mouse/day) | | | | | | | |
| | | day 9~ | day 10~ | day 11~ | day 12~ | day 14~ | day 15~ | day 16~ | day 17~ |
| Group | N | day 10 | day 11 | day 12 | day 13 | day 15 | day 16 | day 17 | day 18 |
| Model | 10 | 6.53 ± | 5.52 ± | 5.94 ± | 5.9 ± | 5.02 ± | 5.68 ± | 5.7 ± | 5.58 ± |
| | | 1.65 | 1.75 | 1.33 | 0.65 | 0.54 | 0.25 | 0.85 | 1.02 |
| Semaglutide | 10 | 6.35 ± | 5.53 ± | 6.31 ± | 5.64 ± | 6.12 ± | 6.46 ± | 6.8 ± | 6.57 ± |
| (10 | | 0.95 | 0.27 | 0.69 | 0.23 | 0.4 | 0.06 | 0.17 | 0.55 |
| nmol/kg) | | | | | | | | | |
| DMS- | 9 | 4.18 ± | 4.63 ± | 4.82 ± | 4.53 ± | 4.85 ± | 3.42 ± | 4.48 ± | 4.3 ± |
| ASD | | 0.08 | 0.86 | 1.58 | 1.68 | 0.66 | 1.07 | 0.51 | 0.4 |
| (1000 | | | | | | | | | |
| mg/kg) | | | | | | | | | |
| AQ-C-a | 9 | 4.59 ± | 4.67 ± | 4.17 ± | 4.14 ± | 4.68 ± | 4.61 ± | 4.64 ± | 4.27 ± |
| (600 | | 0.41 | 0.89 | 0.78 | 0.34 | 0.62 | 0.58 | 0.57 | 0.47 |
| mg/kg) | | | | | | | | | |

| | | Food Intake (g/mouse/day) | | | | | | |
|---|---|---|---|---|---|---|---|---|
| | | day 18~ | day 19~ | day 21~ | day 22~ | day 23~ | day 24~ | day 25~ |
| Group | N | day 19 | day 20 | day 22 | day 23 | day 24 | day 25 | day 26 |
| Model | 10 | 5.71 ± | 5.55 ± | 6.39 ± | 6.52 ± | 7.17 ± | 6.7 ± | 5.97 ± |
| | | 0.78 | 1.51 | 1.4 | 1.36 | 2.05 | 1.73 | 1.44 |
| Semaglutide | 10 | 5.96 ± | 6.54 ± | 7.01 ± | 5.8 ± | 6.72 ± | 6.82 ± | 6.73 ± |
| (10 | | 0.17 | 0.37 | 0.18 | 0.42 | 0.2 | 0.11 | 0.41 |
| nmol/kg) | | | | | | | | |
| DMS- | 9 | 4.53 ± | 4.67 ± | 4.3 ± | 5.09 ± | 4.63 ± | 4 ± | 4.22 ± |
| ASD | | 0.24 | 0.66 | 1.05 | 0.92 | 0.72 | 0.14 | 0.8 |
| (1000 | | | | | | | | |
| mg/kg) | | | | | | | | |
| AQ-C-a | 9 | 4.08 ± | 5.27 ± | 4.29 ± | 4.85 ± | 4.82 ± | 4.74 ± | 4.49 ± |
| (600 | | 0.62 | 2.22 | 0.33 | 0.18 | 0.2 | 0.9 | 0.74 |
| mg/kg) | | | | | | | | |

Note:
Animals in G3-4 and G4-8 died on Day 27 and Day 22, respectively. Renal cysts were visible upon dissection.

Experimental results demonstrate that after four weeks of consecutive daily oral gavage (once a day), the test substances DMS-ASD at 1000 mg/kg and AQ-C-a at 600 mg/kg significantly reduced both the 16-hour fasting blood glucose and random blood glucose levels in db/db mice. They also enhanced the glucose tolerance capacity of db/db mice and exhibited a tendency to decrease their glycosylated hemoglobin HbA1c levels, body weight, and food intake. However, DMS-ASD at 1000 mg/kg and AQ-C-a at 600 mg/kg did not significantly increase insulin secretion in db/db mice. Further research is needed to explore their hypoglycemic mechanism. Dimyricetin-based diselenide holds great promise for pharmaceutical applications in type II diabetes.

What claimed is claimed:

1. The application of dimyricetin diselenide with the molecular structure as shown in (1) in the treatment of Type II diabetes mellitus is characterized by its use in the preparation of a medicament for treating Type II diabetes mellitus.

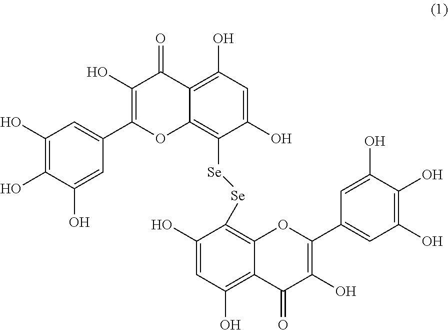 [SMILES: O=c1c(O)c(-c2cc(O)c(O)c(O)c2)oc2c([Se][Se]c3c(O)cc(O)c4c(=O)c(O)c(-c5cc(O)c(O)c(O)c5)oc34)c(O)cc(O)c12]

2. The pharmaceutical application of dimyricetin diselenide in type II diabetes according to claim 1 is characterized in that the dimyricetin diselenide is either the compound itself or a solid dispersion formulation of it.

3. The pharmaceutical application of dimyricetin diselenide in type II diabetes according to claim 2 is characterized in that the solid dispersion formulation of dimyricetin diselenide comprises dimyricetin diselenide and a carrier, wherein the carrier is polyvinylpyrrolidone K30.
